SEQUENCE LISTING (2 sequences)

SEQ ID NO: 1 - protein, 5 aa, synthetic construct
HFRAN

SEQ ID NO: 2 - protein, 8 aa, synthetic construct
KNVWCMDS

(19) United States
(12) Patent Application Publication
(10) Pub. No.: US 2026/0285900 A1
(43) Pub. Date: Sep. 24, 2026

(54) Chemical Carbonylation of Arginine in Peptides and Proteins

(71) Applicant: Emory University, Atlanta, GA (US)

(72) Inventor: Monika Raj, Atlanta, GA (US)

(21) Appl. No.: 19/571,180

(22) Filed: Mar. 18, 2026

Related U.S. Application Data
(60) Provisional application No. 63/773,928, filed on Mar. 18, 2025.

(51) Int. Cl. C07K 1/13; G01N 33/58

(52) U.S. Cl. CPC C07K 1/13; G01N 33/582

(57) ABSTRACT

Disclosed herein are methods of oxidizing a guanidine group comprising contacting a compound or peptide having a guanidine group with a 9,10-phenanthrenequinone or derivative providing compound with an aldehyde group in place of the guanidine group. In certain embodiments, the guanidine group is an arginine amino acid within a protein. In certain embodiments, this disclosure relates to compositions comprising 9,10-phenanthrenequinone derivatives as provided herein.

### CROSS-REFERENCE TO RELATED APPLICATIONS

This application claims the benefit of U.S. Provisional Application No. 63/773,928 filed Mar. 18, 2025. The entirety of this application is hereby incorporated by reference for all purposes.

### STATEMENT REGARDING FEDERALLY SPONSORED RESEARCH OR DEVELOPMENT

This invention was made with government support under HG012941 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 24142US.xml. The XML file is 2,537 bytes, was created on Mar. 17, 2026, and is being submitted electronically via the USPTO patent electronic filing system.

### BACKGROUND

Protein carbonylation, the oxidation of amino acids to carbonyl moieties, is an indicator of oxidative stress and is associated with diseases such as Alzheimer's disease, chronic kidney disease, cancer, diabetes, and fibrosis. The chemoselective incorporation of an arginine carbonylation post-translational modification (PTM) within proteins is sparse because arginine has poor nucleophilicity and resistant to chemical oxidation. Thus, there is a need to identify methods of labeling arginine.

Patthy et al. report the reversible modification of arginine residues. J Biol Chem, 1975, 250(2):557-64.

Shih et al. report dibenzocyclooctendiones as arginine-selective chemical labeling reagents. Org Lett, 2022, 24, 4694-4698.

Smith et al. report a fluorometric method for the determination of arginine using 9,10-phenanthrenequinone. Anal Biochem, 1978, 90(1):246-55.

Wanigasekara et al. report the evaluation of chemical labeling methods for identifying functional arginine residues of proteins by mass spectrometry. Anal Chim Acta, 2016, 935:197-206.

Yamada et al. report phenanthrenequinone as an analytical reagent for arginine and other monosubstituted guanidines. Biochim Biophys Acta, 1966, 130(2):538-40.

References cited herein are not an admission of prior art.

### SUMMARY

In certain embodiments, this disclosure relates to methods of oxidizing a guanidine group comprising contacting a compound or peptide having a guanidine group with a 9,10-phenanthrenequinone or derivative providing compound with an aldehyde group in place of the guanidine group. In certain embodiments, the guanidine group is an arginine amino acid within a protein. In certain embodiments, the protein sequence is a peptide of 3 or more amino acids. In certain embodiments, the protein sequence is greater than 4 or 5 amino acids. In certain embodiments, this disclosure relates to compositions comprising 9,10-phenanthrenequinone derivatives as provided herein.

In certain embodiments, this disclosure relates to methods of oxidizing a guanidine group comprising contacting a compound having a guanidine group with a 9,10-phenanthrenequinone or derivative and base such as a potassium phosphate base providing compound with an aldehyde group in place of the guanidine group. In certain embodiments, this disclosure relates to compositions comprising 9,10-phenanthrenequinone derivatives as provided herein.

In certain embodiments, this disclosure relates to methods of oxidizing a guanidine group comprising contacting a compound having a guanidine group with a 9,10-phenanthrenequinone or derivative and 1,8-diazabicycloundec-7-ene (DBU) providing compound with an aldehyde group in place of the guanidine group.

In certain embodiments, the compound with the aldehyde group is contacted with a labeling agent comprising an amine group. In certain embodiments, the amine group is a primary amine group, secondary amine group, hydroxy amine group, hydrazine group, aniline amine group. In certain embodiments, the labeling agent comprises an aromatic molecule, a fluorescent agent, a ligand, biotin, a receptor, enzyme, an antigen, radioisotope, or an antibody.

In certain embodiments, this disclosure relates to methods of oxidizing a guanidine group comprising contacting a compound having a guanidine group with a 9,10-phenanthrenequinone or derivative providing compound with an aldehyde group in place of the guanidine group. In certain embodiments, the 9,10-phenanthrenequinone or derivative has the Formula I,

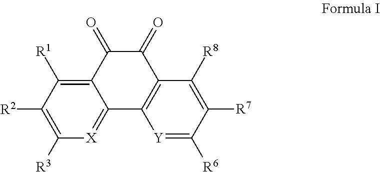

or salts thereof, wherein
X is N or CR⁴;
Y is N or CR⁵;
R¹, R², R³, R⁴, R⁵, R⁶, R⁷ and R⁸ are individually selected from hydrogen, C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein each R¹, R², R³, R⁴, R⁵, R⁶, R⁷ and R⁸ are optionally substituted with one or more, the same or different, R⁹; and
R⁹ is C₁-10 alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein R⁹ is optionally substituted with one or more, the same or different, R¹⁰; or
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, isopropoxycarbonyl, tert-butoxycarbonyl, benzoyl, benzyl, carbocyclyl, aryl, or heterocyclyl.

In certain embodiments, R⁶ is carboxy. In certain embodiments, X is N and Y is N. In certain embodiments, X is CH and Y is N. In certain embodiments, X is N and Y is CH. In certain embodiments, X is CH and Y is CH. In certain embodiments, R⁴ is C-nitro. In certain embodiments, R⁷ is nitro. In certain embodiments, R³ is halogen. In certain embodiments, R³ is carboxy. In certain embodiments, R⁴ is C-nitro. In certain embodiments, R⁷ is amino. In certain embodiments, R⁴ is amino.

In certain embodiments, the compound is 9,10-phenanthrenequinone-3-carboxylic acid, 2,5-dinitrophenanthrene-9,10-dione, or 1,10-phenanthroline-5,6-dione, derivative or salt thereof.

### BRIEF DESCRIPTION OF THE SEVERAL VIEWS OF THE DRAWINGS

FIG. 1 illustrates the carbonylation of arginine in natural products such as proteins, cells, and cell lysates.

FIG. 2A illustrates the reaction of phenanthrenequinone with arginine under basic conditions in a mixture of acetonitrile and water providing a guanidine fluorophore and imine compound 3a, i.e. which can hydrolyse to form an aldehyde as illustrated in FIG. 2B.

FIG. 2B illustrates the formation of glutamate-5-semialdehyde by the reaction of carboxylate 9,10-phenanthrenequinone (2b) with L-Arg in a peptide having SEQ ID NO: 1 and providing certain reaction products.

FIG. 2C shows a table of data for the reaction illustrated in FIG. 2B using various reagents and reaction conditions providing for optimized reaction conditions to obtain high conversion to glutamate-5-semialdehyde by varying bases.

FIG. 3 shows data on the evaluation of substituted 9,10-phenanthrenequinone analogs on the arginine carbonylation reaction. Screening of varying 9,10-phenanthrenequinone analogs with EWG and ERG for the modification of Arg to aldehyde on a model peptide 1b. nr=no reaction, nr*=no reaction under optimized conditions but reaction condition was modified to show the generation of the fluorophore byproduct.

FIG. 4 illustrates a conversion to the corresponding glutamate-5-semialdehyde 3d and anthracene-HRW, 3d′.

FIG. 5 illustrates fluorescence tagging of arginine in proteins and the proteome. Carbonylation of proteins (myoglobin, lysozyme human, and lysozyme chicken) was performed followed by oxime formation with hydroxylamine-647 fluorophore. The products were analyzed by in-gel fluorescence.

FIG. 6 illustrates and provides data on chemoproteomics profiling of arginine residues prone to oxidation in cell lysates. T-47D breast cancer cell lysates were treated with reagents at low (50 μM), medium (100 μM), high (250 μM), and superhigh doses (1000 μM) of carboxylate-substituted 9,10-phenanthrenequinone 2b in 1:4 ACN:water, followed by Glu-C digestion for LC-MS/MS analysis. Analysis identified the modification of 250 unique peptides at 50 μM, 310 unique peptides at 100 μM, 473 unique peptides at 250 μM, and 3401 unique peptides at 1000 μM. Seventeen peptides were observed in the control sample (0 μM). Unique peptides for all concentrations were normalized using the control sample (0 M). Analysis of probe 2b modified peptides to identify proteins with hyperreactive arginine residues led to the identification of 8 proteins with arginine sites susceptible to carbonylation. Sequence motif analysis of carbonylated arginine sites clearly identified a significant distribution of flexible and turn inducing residues such as glycine, proline, and alanine followed by glutamic acid.

FIG. 7 illustrates the incorporation of various post-translational modifications (PTMs) after carbonylation of arginine by labeling of the generated aldehyde moiety. Incorporation of various PTMs is implemented by reductive amination of carbonylated arginine, e.g., ornithine generated with ammonia, monomethylation of ornithine generated with N-methylamine, dimethylation of ornithine with N,N-dimethylamine, dimethylation of ornithine with N,N-diethylamine, and N-methylpropargylation of ornithine with N-methylpropargylamine.

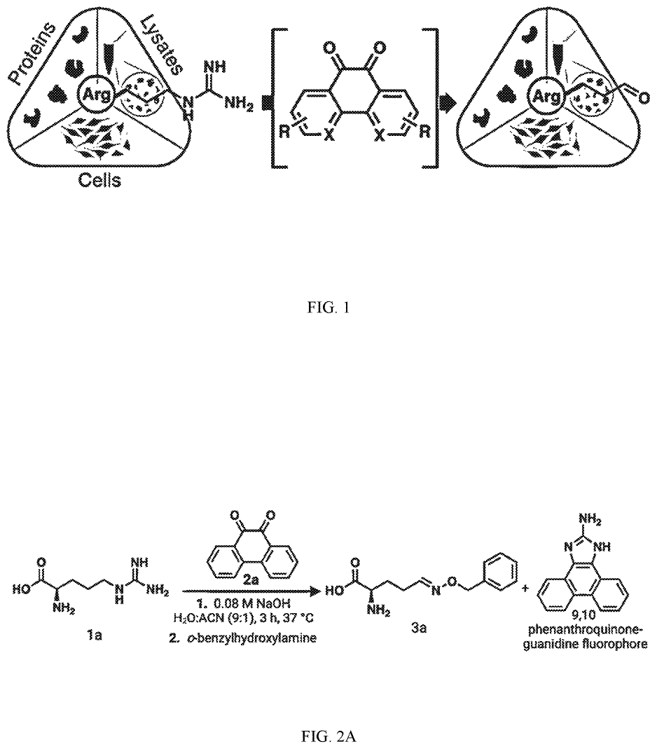

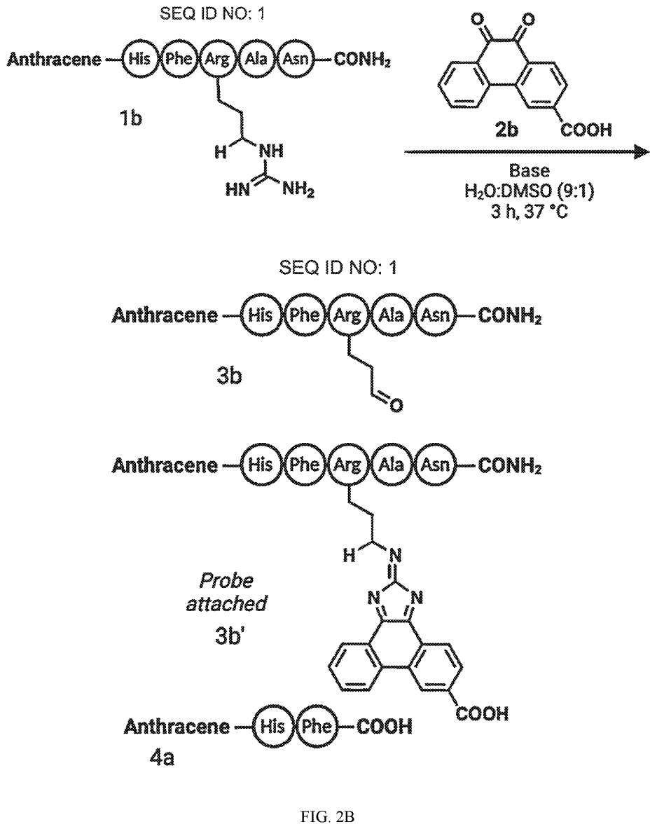

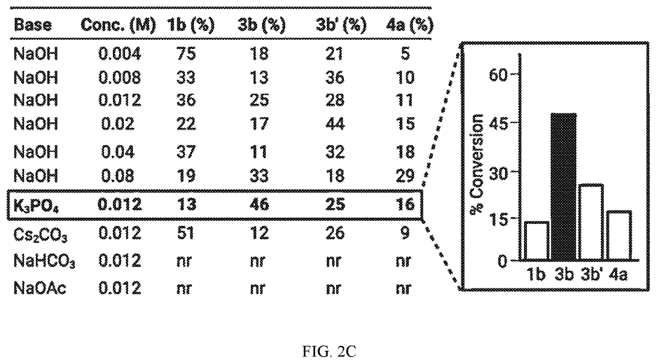

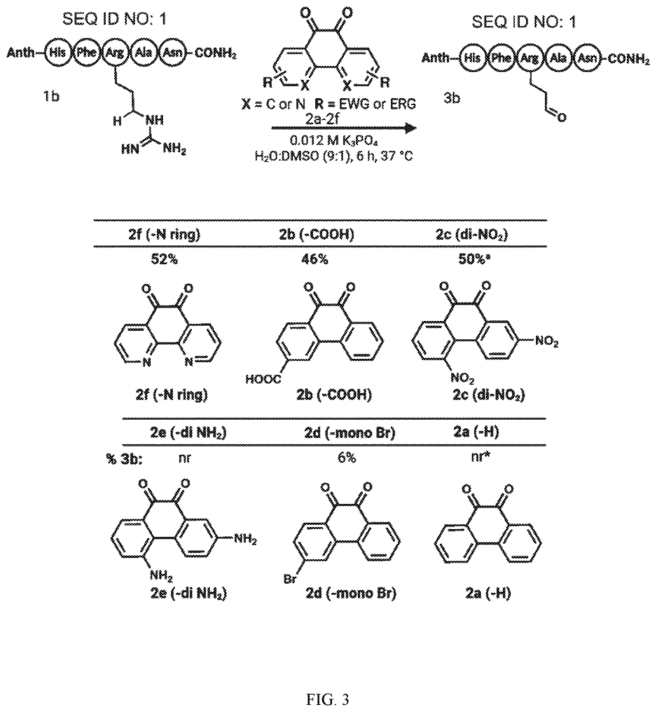

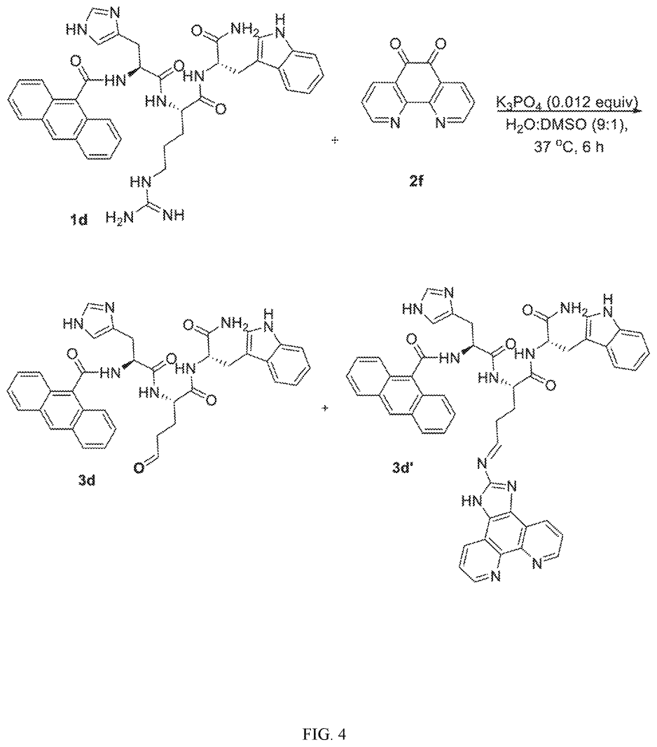

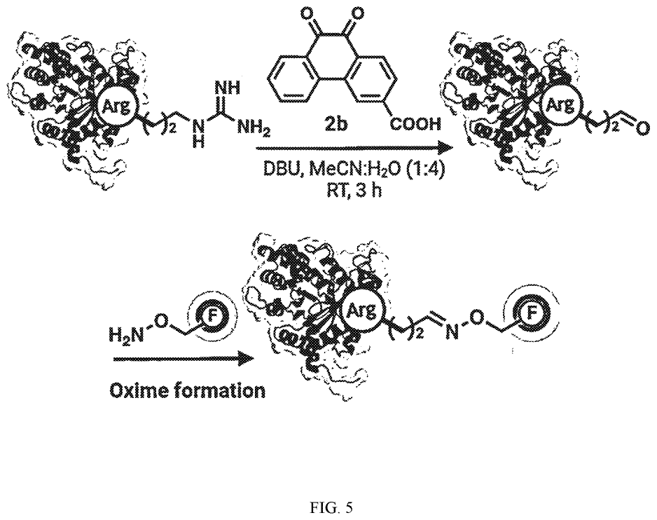

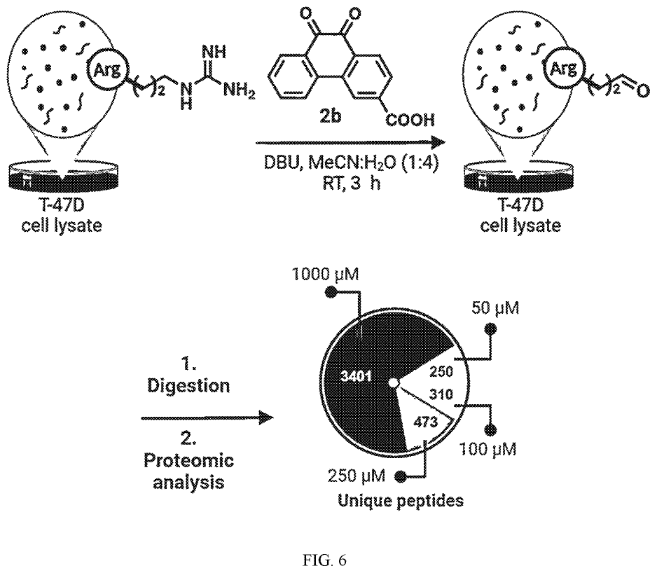

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims or as amended during prosecution.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure indicates that it is an example and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “protein,” “peptide,” and “polypeptide” refer to compounds comprising amino acids joined via peptide bonds and are used interchangeably. As used herein, an “amino acid sequence” refers to an amino acid sequence of a protein molecule. An “amino acid sequence” can be deduced from the nucleic acid sequence encoding the protein. However, terms such as “peptide” or “protein” are not meant to limit be limited to natural amino acids. The term includes naturally and non-naturally derived material optionally having naturally or non-naturally occurring amino acids and modifications such as, substitutions, glycosylations, and addition of hydrophilic or lipophilic moieties. In certain embodiments, the protein/peptide/polypeptide comprises more than three, four, five, six, seven, eight, nine, or ten amino acids.

The terms, “cell culture” or “growth medium” or “media” refers to a composition that contains components that facilitate cell maintenance and growth through protein biosynthesis, such as vitamins, amino acids, inorganic salts, a buffer, and a fuel, e.g., acetate, succinate, a saccharide and/or optionally nucleotides. Typical components in a growth medium include amino acids (histidine, isoleucine, leucine, lysine, methionine, phenylalanine, threonine, tryptophan, valine and others); vitamins such as retinol, carotene, thiamine, riboflavin, niacin, biotin, folate, and ascorbic acid; carbohydrate such as glucose, galactose, fructose, or maltose; inorganic salts such as sodium, calcium, iron, potassium, magnesium, zinc; serum; and buffering agents. Additionally, a growth media may contain phenol red as a pH indication. Components in the growth medium may be derived from blood serum, or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Other contemplated components contemplated in a growth medium include ammonium metavanadate, cupric sulfate, manganous chloride, ethanolamine, and sodium pyruvate. Minimal Essential Medium (MEM) is a term of art referring to a growth medium that contains calcium chloride, potassium chloride, magnesium sulfate, sodium chloride, sodium phosphate and sodium bicarbonate), essential amino acids, and vitamins: thiamine (vitamin B1), riboflavin (vitamin B2), nicotinamide (vitamin B3), pantothenic acid (vitamin B5), pyridoxine (vitamin B6), folic acid (vitamin B9), choline, and myo-inositol (originally known as vitamin B8). Various growth mediums are known in the art. Dulbecco's modified Eagle's medium (DMEM) is a growth medium which contains additional components such as glycine, serine, and ferric nitrate with increased amounts of vitamins and amino acids.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—,
—C(OH)Rₙ, —C(OH)(OH)—, —C(OH)H, —C(Hal)Rₙ—, —C(Hal)(Hal)-, —C(Hal)H—, —C(N₃)Rₙ—,
—C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or —Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, glycol, and aromatic groups.

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as a ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabelled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ¹⁸F, ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

Hydrophilic polymers contain polar or charged functional groups, rendering them soluble in water. Examples include polyethylene glycol, polylactides, polyglycolide, poly(ε-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly[oligo(ethylene glycol) vinyl ether], poly{[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). Typically, PEGs comprise the following structure “—(OCH₂CH₂)n-” where (n) is 2 to 4000.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

For any element disclosed herein expressly, by chemical formula, by inference, or by a single letter designation, the element is contemplated to include that element in its natural abundance or versions accessible in enriched forms in excess of natural abundance using standard synthetic techniques. For example, “H” or “hydrogen” refers to the hydrogen element or versions accessible as enriched in excess of natural abundance for common isotopes thereof, e.g., deuterium, ²H, or tritium ³H. Similarly, enriched halogen isotopes are contemplated, e.g., ¹⁸F, ¹⁹F, or ⁷⁶Br.

As used herein, “alkyl” means a noncyclic straight chain or branched, unsaturated or saturated hydrocarbon such as those containing from 1 to 10 carbon atoms, typically 1 to 6 carbon atoms. Within any embodiments, herein alkyl may refer to an alkyl with 1 to 6 carbons (C₁₋₆alkyl). Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen, and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen, and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bride (i.e., —(C═O)alkyl).

“Alkoxycarbonyl” refers to an alkyl as defined above attached through a carboxy bridge (i.e., —(C═O)Oalkyl.

“Alkylcarbamoyl” refers to an alkyl as defined above attached through a carbonyl bridge (i.e., —(C═O) NHalkyl).

“Alkanoyl” refers to an alkyl as defined above attached through a carbonyl bridge (i.e., —(C═O)alkyl).

“Glycol” refers to an alkyl group substitute with terminal oxygen atoms, and the term is intended to include repeating glycol units, e.g., polyethylene glycol —O(CH₂CH₂O)ₙX, wherein X is typically an H, alkyl, alkoxy, halogenated alkoxy, or alkanoyl, and n is typically 1-50, 1-100, or 1-1,000.

The terms “halogen” and “halo” refer to fluorine, chlorine, bromine, and iodine.

The term “halogenated alkyl” refers to an alkyl partially or entirely substituted with halogens, e.g., CF₃.

The term “substituted” refers to a molecule wherein at least one hydrogen atom is replaced with a substituent. When substituted, one or more of the groups are “substituents.” The molecule may be multiply substituted. In the case of an oxo substituent (“—O”), two hydrogen atoms are replaced. Example substituents within this context may include halogen, hydroxy, alkyl, alkoxy, halogenated alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b},
—NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b},
—OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, halogenated alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In certain embodiment the salts are conventional nontoxic pharmaceutically acceptable salts including the quaternary ammonium salts of the parent compound formed, and non-toxic inorganic or organic acids. Contemplated salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

### Methods of Oxidizing a Guanidine Group

In certain embodiments, this disclosure relates to methods of oxidizing a guanidine group comprising contacting a compound having a guanidine group with a 9,10-phenanthrenequinone or derivative and a base such as DBU or potassium phosphate base providing peptides or compounds with an aldehyde group in place of the guanidine group. In certain embodiments, the guanidine group is an arginine amino acid within a protein.

In certain embodiments, the protein sequence is greater than 5 amino acids. In certain embodiments, the protein sequence is greater than 10 amino acids. In certain embodiments, the protein sequence is greater than 15 amino acids. In certain embodiments, the protein sequence is greater than 20 amino acids. In certain embodiments, the protein sequence is greater than 30 amino acids. In certain embodiments, the protein sequence is greater than 40 amino acids.

In certain embodiments, this disclosure relates to methods of oxidizing a guanidine group comprising contacting a sample comprising a peptide having a guanidine group with a 9,10-phenanthrenequinone or derivative and a base, e.g., DBU or potassium phosphate base providing compound with an aldehyde group in place of the guanidine group. In certain embodiments, the peptide with an aldehyde group is contacted with a labeling agent comprising an amine group.

In certain embodiments, the labeling agent comprising an amine group is within a primary amine group, secondary amine group, hydroxy amine group, hydrazine group, or aniline amine group. In certain embodiments, the labeling agent comprises an aromatic molecule, a fluorescent agent, a ligand, biotin, a receptor, enzyme, an antigen, radioisotope, or an antibody.

In certain embodiments, the amine group can be conjugated to a solid support such as a particle. In certain embodiments, this disclosure relates to methods of purifying a compound or peptide comprising a guanidine group in a sample comprising contacting the sample with 9,10-phenanthrenequinone or derivative as reported herein and a base providing compound with an aldehyde group in place of the guanidine group, e.g. providing peptide comprising an aldehyde group in place of the guanidine amino acid in the sample. In certain embodiments, methods of purifying the compound/peptide with the aldehyde group is by mixing the aldehyde containing compound/peptide with a secondary labeled compound comprising an amine to form a labeled imine compound. In certain embodiments, the formed imine compound comprises an alkynyl group. In certain embodiments, the imine compound is contacted with a labeled triazene compound/peptide to form a triazole label. In certain embodiments, imine compound comprising an alkynyl group is contacted with to a triazene group conjugated to a solid support under conditions such that the alkynyl group reacts with the triazene group on the solid support to form a triazole linkage providing a solid support conjugated to the triazene. In certain embodiments, methods entail further purifying or washing the solid support to remove unreacted material from the sample providing a purified solid support conjugated to the triazene. In certain embodiments, methods further comprise contacting the purified solid support conjugated to the triazole with acid conditions providing a composition with a purified compound or peptide release from the solid support.

### Derivatives of 9,10-phenanthrenequinone

In certain embodiments, it is contemplated that 9,10-phenanthrenequinone derivative can be any a compound containing a substitution or heteroatom substitution.

In certain embodiments, the 9,10-phenanthrenequinone or derivative has the following formula I,

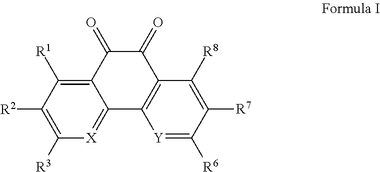

or salt thereof, wherein
   X is N or CR⁴;
   Y is N or CR⁵;
R¹, R², R³, R⁴, R⁵, R⁶, R⁷ and R⁸ are individually selected from hydrogen, C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein each R¹, R², R³, R⁴, R⁵, R⁶, R⁷ and R⁸ are optionally substituted with one or more, the same or different, R⁹; and
R⁹ is C₁-10 alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein R⁹ is optionally substituted with one or more, the same or different, R¹⁰; or
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, isopropoxycarbonyl, tert-butoxycarbonyl, benzoyl, benzyl, carbocyclyl, aryl, or heterocyclyl.

In certain embodiments, R⁶ is carboxy. In certain embodiments, X is N and Y is N. In certain embodiments, X is CH and Y is N. In certain embodiments, X is N and Y is CH. In certain embodiments, X is CH and Y is CH. In certain embodiments, R⁴ is C-nitro. In certain embodiments, R⁷ is nitro. In certain embodiments, R³ is halogen. In certain embodiments, R³ is carboxy. In certain embodiments, R⁴ is C-nitro. In certain embodiments, R⁷ is amino. In certain embodiments, R⁴ is amino.

In certain embodiments, R³ is carboxy. In certain embodiments, the compound is 9,10-phenanthrenequinone-3-carboxylic acid or salt thereof.

In certain embodiments, X is N and Y is N.

In certain embodiments, R⁷ is nitro.

In certain embodiments, R³ is halogen.

In certain embodiments, the 9,10-phenanthrenequinone derivative is selected from the compounds:

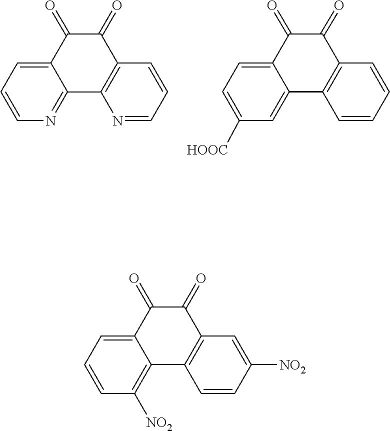 [SMILES: O=C1C(=O)c2cccnc2-c2ncccc21 ; O=C(O)c1ccc2c(c1)-c1ccccc1C(=O)C2=O ; O=C1C(=O)c2cccc([N+](=O)[O-])c2-c2ccc([N+](=O)[O-])cc21]

salts, esters, amides or derivatives thereof.

### Chemical Carbonylation of Arginine in Peptides and Proteins

Disclosed herein is a chemical methodology aimed at generating glutamate-5-semialdehyde from arginine residues within peptides and proteins. This chemical approach capitalizes on the inherent weak nucleophilicity and oxidative properties of arginine. The application of this strategy is used to selectively introduce both natural and unnatural post-translational modifications (PTMs) in a targeted manner. This methods has benefits as a rapid, robust, and highly selective technique, facilitating chemoproteomic profiling of arginine sites prone to forming glutamate-5-semialdehyde PTMs within the human proteome, and can be used as a versatile platform for uncovering microenvironments that are susceptible to undergoing arginine carbonylation PTMs, enabling the understanding of the effect of oxidative stress on arginine in proteins and the impact of these PTMs on cellular processes.

Protein carbonylation, the oxidation of amino acids to carbonyl moieties, is an indicator of oxidative stress and is associated with diseases such as Alzheimer's disease, chronic kidney disease, cancer, diabetes, and fibrosis. Arginine is abundant in proteins. Protein carbonylation of arginine can occur through nonenzymatic mechanisms such as direct exposure to reactive oxygen species (ROS) or metal-catalyzed oxidation (MCO) of arginine in the presence of ROS and reduced metal ions to generate glutamate-5-semialdehyde. This disclosure relates to selective chemical methods for incorporating glutamate-5-semialdehyde into peptides and proteins in order to facilitate the dentification of arginine residues on proteins that are more prone to carbonylation and elucidate their role in modulating interactions with other proteins. The chemical incorporation of arginine carbonylation has been challenging due to the low nucleophilicity and resistance of its guanidine group to chemical oxidation. It has been discovered that one can oxidize arginine by direct MCO in the presence of ROS by the reaction of guanidine with dicarbonyl compounds. One can utilize 9,10-phenanthrenequinone for the carbonylation of arginine residues in peptides and proteins to form glutamate-5-semialdehyde. This method facilitates rapid protein diversification, marking a significant step in the selective incorporation of arginine carbonylation within complex biological environments. The application of this arginine carbonylation strategy has led to the identification of certain protein targets that are more susceptible to arginine oxidation in the human proteome. Chemoproteomics revealed sequence motifs of arginine residues that are prone to carbonylation within the human proteome. This chemical strategy has utility in generating cellular models of arginine carbonylation; thereby, providing a chemical platform for understanding its effect on cellular components. As this method introduces reactive aldehyde groups onto peptides and proteins one can functionalize the peptides through aldehyde-specific reactions such as hydroxylamine and reductive amination.

### Development of Chemical Carbonylation of Arginine

Arginine carbonylation using 9,10-phenanthrenequinone derivatives to selectively modify arginine (Arg) residues in peptides and proteins to glutamate-5-semialdehyde was investigated. Commencing with the treatment of L-Arg 1a with 9,10-phenanthrenequinone 2a in aqueous solution H2O:ACN (9:1) and 0.08 M NaOH at 37° C. for 3 h, the formation of glutamate-5-semialdehyde was observed. The aldehyde was converted to oxime 3a through reaction with benzyl hydroxylamine and subsequently isolated. The reaction also produced a 9,10-phenanthrenequinone-guanidine fluorophore, causing the reaction mixture to turn purple. Subsequently, the reaction was carried out with a small molecule benzyl guanidine with 9,10-phenanthrenequinone analogue 2b in ACN:H2O (9:1) and 0.08 M NaOH at 37° C. for 3 h and the formation of benzaldehyde (70% yield) was observed along with the formation of 9,10-phenanthrenequinone-guanidine fluorophore. It is contemplated that the reaction mechanism for the guanidine carbonylation involves nucleophilic addition of arginine to diketones of 9,10-phenanthrenequinone, generating imines. It is contemplated that the aromatic rings of 9,10-phenanthrenequinone enhance conjugation, consequently lowering the pKa of the alpha-carbon proton adjacent to guanidine. This decrease in pKa leads to deprotonation of the alpha-carbon proton, followed by isomerization to create a new imine that is hydrolyzed to create the 9,10-phenanthrenequinone-guanidine fluorophore and glutamate-5-semialdehyde.

To adapt this reaction for carbonylation of Arg on proteins, the water soluble 9,10-phenanthrenequinone-3-carboxylic acid was investigated under aqueous conditions H2O: DMSO (9:1) using a model peptide anthracene-HFRAN (SEQ ID NO: 1, 1b). The peptide was labeled with anthracene to increase its visibility on HPLC at 220 nm due to the high absorbance of the 9,10-phenanthrenequinone and the corresponding fluorophore obtained by the reaction of 9,10-phenanthrenequinone with the Arg side chain. By varying the amounts of sodium hydroxide as a base (from 0.004 to 0.08 M), the formation of glutamate-5-semialdehyde product anthracene-HFR—(CHO)AN 3b was observed, alongside the formation of the probe-attached anthracene-HFR(PQ)AN product 3b′ and the peptide cleavage product 4a. It is contemplated that the cleavage product arises from the formation of a pyrollinium-like intermediate between the amide backbone chain and the aldehyde handle obtained from the Arg side chain, leading to hydrolysis of the backbone amide. To enhance conversion to the desired glutamate-5-semialdehyde peptide product 3b, various bases such as K₃PO₄, Cs₂CO₃, NaHCO₃, and NaOAc, were tested. The maximum conversion (46%) occurred with 0.012 M K₃PO₄. This data indicates the base in deprotonating the proton of the a carbon adjacent to the guanidine ring. Significant cleavage of the C-terminal Arg residue (<13%) was not observed under the reaction conditions which is in contrast to the results reported in Prosser et al. entitled C-terminal arginine-selective cleavage of peptides as a method for mimicking carboxypeptidase b. Org. Lett. 2023, 25, 6206-6210. To further enhance the conversion to the desired glutamate-5-semialdehyde product 3b within the peptide 1b, analogs of 9,10-phenanthrenequinone (2a-2f) were examined that contained electron-withdrawing (EWG) groups (mono-COOH; 2b, di-NO2; 2c) and electron-releasing (ERG) groups (mono-Br; 2d, di-NH2; 2e), including the phenanthroline derivative 2f, where the CH groups in the hydrocarbon at positions 4 and 5 of 9,10-phenanthrenequinone are replaced with nitrogen atoms. Probes 2c and 2e were synthesized in a multistep process.

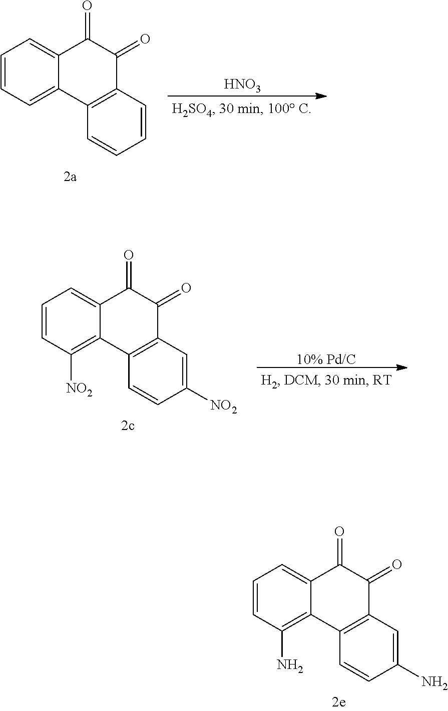 [SMILES: O=C1C(=O)c2ccccc2-c2ccccc21 ; "HNO3" ; O=C1C(=O)c2cccc([N+](=O)[O-])c2-c2ccc([N+](=O)[O-])cc21 ; "10% Pd/C" ; Nc1ccc2c(c1)C(=O)C(=O)c1cccc(N)c1-2]

These analogs were tested by adding one equivalent to a model peptide anthracene-HFRAN 1b in a 9:1 H₂O:DMSO solution with 0.012 M of K₃PO₄ at 37° C. for 6 h, followed by HPLC and MS analysis. A visible change in the color of the reaction mixtures was observed, plausibly due to the formation of 9,10-phenanthrenequinone-guanidine fluorophore during the hydrolysis of imine that generates glutamate-5-semialdehyde 3b. The experiments demonstrated an increased conversion to the glutamate-5-semialdehyde product anthracene-HFR—(CHO)AN 3b (52%) with 9,10-phenanthroline-5,6-dione 2f, which further increased to 71% in 10 h, compared to no conversion with the unsubstituted 9,10-phenanthrenequinone 2a and 6% conversion with mono-Br (2d) derivative. Evaluation of 9,10-phenanthrenequinone-3-carboxylic acid 2b led to decent conversion to the desired glutamate-5-semialdehyde product anthracene-HFR(CHO)AN 3b (46%). Screening of phenanthrenequinone analogue 2c with two nitro substituents led to the immediate degradation under the reaction conditions. However, evaluation of the di-nitro probe 2c in basic conditions of 0.01 M NaOH hydroxide led to 50% formation of 3b in 3 h. These results suggest an increase in the reactivity of phenanthrenequinone analogs (2b, 2c, and 2f) substituted with electron-withdrawing groups (EWGs) toward arginine. These observations indicated an increased reactivity of the diketone moiety in EWG analogs with the guanidinium group of arginine. The experimental findings indicate an increased reactivity of electron withdrawing analogs over electron releasing substituents.

### Chemoselectivity Studies of Carbonylation Reaction

Chemoselectivity studies unveiled the specificity of the carbonylation reaction in producing aldehyde products exclusively with Arg, as evidenced by the reaction of 2b with peptides 1c, anthracene-KNVWCMDS (SEQ ID NO: 2), and anthracene-HRW, containing different reactive amino acids with high nucleophilicity and oxidation potential (K, N, W, C, M, D, S) as compared to Arg. A conversion of 58% to the corresponding glutamate-5-semialdehyde 3d anthracene-HRW was observed under the reaction conditions as analyzed by HPLC and MS. Oxidation of Met or Cys under the reaction conditions was not observed.

### Selective Chemical Carbonylation of Arginine in Proteins

Expanding experiments to protein carbonylation, optimization studies were commenced using myoglobin (Mb), which contains two arginine residues by reacting with the water-soluble electron-withdrawing probe 2b. Initial treatment of myoglobin (240 μM) with 2b (480 μM, 2 equiv) in a 1:9 ACN:H2O in 0.08 M NaOH primarily modified one Arg to glutamate-5-semialdehyde (75% conversion) within 5 h, as confirmed by LCMS. A similar observation was made when the reaction was evaluated using 13 mM organic base (DBU), yielding 72% conversion to a single modification of arginine. Subsequent MS/MS analysis of modified myoglobin identified R31 as the preferred modification site. Although two of the arginines present in myoglobin are surface exposed, only one was preferentially carbonylated, suggesting a differential propensity of various arginine residues to undergo oxidation.

Using 9,10-phenanthrenequinone analogue 2b and 0.08 M NaOH, reactions were conducted with additional proteins, including aprotinin and ubiquitin. Aprotinin, with six arginine residues and three disulfides, exhibited full conversion to two Arg carbonylations. MS/MS analysis revealed R39 and R53 as sites of modifications on aprotinin. A 68% conversion of Arg to glutamate-5-semialdehyde was observed with ubiquitin, resulting primarily in a single modification of one arginine, along with minor modification of the second arginine out of the four arginines present in ubiquitin. Subsequent MS/MS analysis of modified and digested ubiquitin identified R42 as the preferred site of modification along with the minor modification of R54. To further demonstrate the scope of the electron withdrawing analogs of 9,10-phenanthrenequinone, Probe 2c in 0.01 M NaOH was utilized for the carbonylation of additional protein substrates with varying 3D structures and molecular weights (14.3-80 kDa) such as RNase, lysozyme chicken, chymotrypsinogen, and apo-transferrin. These experiments exhibited 70 to >95% modification of arginine to glutamate-5-semialdehyde with excellent selectivity, even at low concentrations of proteins (70 μM). The analysis of the intact mass spectra of carbonylated proteins showed no breakage of the disulfides, further confirming the highly chemoselective nature of our carbonylation platform. By adjusting the reaction conditions, homogeneous modification to a single aldehyde product can be achieved on varying proteins of different sizes and molecular weights such as myoglobin, ubiquitin, aprotinin, and cytochrome C. These results demonstrate an ability to perform the carbonylation reaction of a wide range of protein substrates.

### Arginine Carbonylation Generates De Novo Intramolecular Imines and Hydrates

Protein carbonylation has been reported to activate various signaling pathways associated with diseases. This is primarily attributed to the alteration of the interactions and structures resulting from the conversion of the positively charged arginine residue to a neutral and reactive aldehyde group. Investigating these changes in interactions due to selective chemical carbonylation of arginine is important for understanding its implications in cellular processes and disease mechanisms. The analysis of chemical carbonylation of arginine in various proteins led to an interesting observation: the formation of imines (−18 Da) and hydrate (+18 Da) derived from glutamate-5-semialdehyde product. This observation indicates a plausible formation of Schiff base between glutamate-5-semialdehyde and a nearby lysine residue and the hydration of the carbonyl group of glutamate-5-semialdehyde. To demonstrate the formation of imine and hydrate glutamate-5-semialdehyde, NMR investigations of small molecule mimics of aldehydes and amines were performed.

Water-soluble 2,4-dihydroxybenzaldehye was incubated with β-alanine in deuterated DMSO-phosphate buffer) mixture for 2 h. Proton NMR analysis revealed the formation of characteristic imine protons that were absent in 2,4-dihydroxybenzaldehye without β-alanine. Additionally, the incubation of butyraldehyde in 20 mM phosphate buffer for 2 hours led to the appearance of characteristic hydrate protons that were absent in DMSO control. Taken together, these observations highlight the potential intra- and inter-cross-linking of lysine residues with glutamate-5-semialdehyde within carbonylated proteins.

### Chemical Carbonylation for Installation of Fluorophores in Proteins and Cell Lysates

The ability to generate glutamate-5-semialdehyde also allows for further diversification of the generated aldehyde modalities with affinity tags and fluorophores. To assess this capability, carbonylated proteins (myoglobin, human lysozyme, and chicken lysozyme) were functionalized with hydroxylamine-647 fluorophores which were analyzed using in-gel fluorescence. The results distinctly displayed fluorescent labeling of carbonylated proteins, with no fluorescence signal observed without treatment with 9,10-phenanthrenequinone-3-carboxylic acid 2b under reaction conditions.

Due to the high labeling efficiency of this method in individual proteins, the applicability of selective labeling of native proteins was applied to complex systems such as the cell lysate. Cell lysate obtained from prostate cancer cells (T-47D) were incubated with 2b and DBU in a dose-dependent manner (100 μM to 1 mM) for 3 h, followed by the attachment of hydroxylamine-647 dye. In-gel fluorescence analysis revealed extensive labeling of Arg on proteins in the cell lysate mixture in the presence of all of the components. No labeling was observed in the control experiments without the carbonylation reagent 2b. The ability to selectively introduce arginine carbonylation in a complex mixture such as cell lysate supports the potential application of this chemical approach for evaluating protein sites that are susceptible to oxidation.

### Arginine Carbonylation Chemoproteomic Profiling

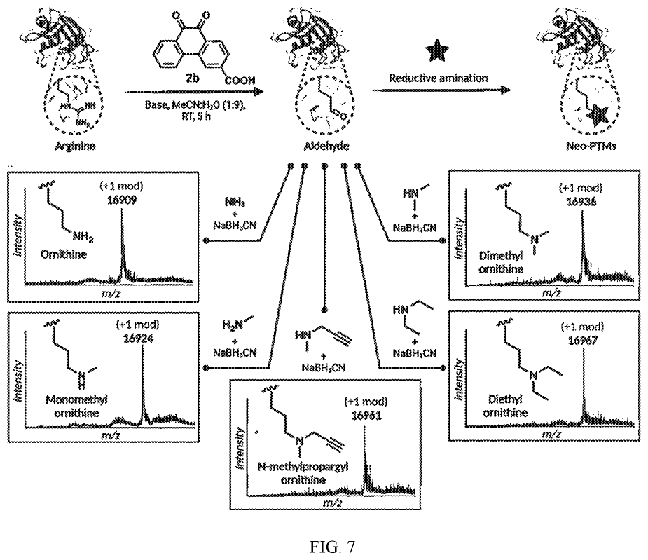

To further demonstrate the application of this chemical strategy for uncovering arginine sites in the proteome that are susceptible to carbonylation, T-47D cell lysates weew incubated with varying concentrations of carboxylate-substituted 9,10-phenanthrenequinone 2b (50 μM to 1 mM), followed by digestion and LC-MS/MS analysis. Analysis revealed a dose dependent conversion of Arg to glutamate-5-semialdehyde in the proteome (250 unique peptides at 50 μM; 310 unique peptides at 100 M; 473 unique peptides at 250 μM; and 3401 unique peptides at 1 mM; FIG. 7a, Figure S13). This result demonstrates the increasing generation of arginine carbonylation products under increased oxidative stress conditions.

A comparison of the carbonylated proteins identified using phenanthrenequinone 2b with previously arginine-carbonylated proteins observed in cells under oxidative stress modified by metal-catalyzed oxidation (MCO) revealed an overlap of approximately 30% (43 out of 144 proteins). It is noted that the cell lines and experimental conditions utilized in these studies are distinct, which may account for the differences in the identified proteins. The major means of generating arginine carbonylation in cells is through metal catalyzed oxidations (MCO) and not enzyme catalysis. Since the mechanism of arginine oxidation in cells under oxidative stress via metal catalyzed oxidation (MCO) parallels the mechanism employed by our phenanthrenequinone probe, it is reasonable to contemplate that the carbonylated sites identified using the probes serve as markers for arginine residues particularly prone to carbonylation.

To confirm the correlation between the methods and the metal catalyzed oxidation (MCO) reaction, attempts were made to modify proteins under MCO reaction conditions. Formation of Arg-to-aldehyde conversions were not observed under any of the reported conditions for modifying Arg to aldehydes in cells. This lack of observable modification underscores the significance of these experiments findings, which represents the first reported method to achieve this selective modification.

Further, the sorting of carbonylated arginine sites in at least 3 concentrations identified 8 proteins possessing arginine sites with an elevated propensity to undergo carbonylation. Most of the modified proteins possess enzymatic, regulatory, and structural functions. The sequence motif analysis of carbonylated arginine sites points to a significant distribution of flexible and turn inducing residues such as glycine, proline, and alanine followed by glutamic acid. It is contemplated that these arginine residues are more prone to carbonylation because the neighboring flexible and turn inducing residues enable glutamic acid to abstract the proton from the Arg thus activating them for the reaction with diketones.

Gene ontology analysis revealed the significant enrichment of carbonylated arginine residues within numerous proteins exhibiting enzymatic, regulatory and structural functions. These observations highlight the encompassing effect of arginine carbonylation on various biological functions and processes. Interestingly, these observations closely correlate with the effect of protein carbonylation on cytoskeletal proteins, extracellular matrix proteins, in addition to ATP binding proteins.

### Carbonylation of Proteins Inside Cells

The ability to modify Arg-containing proteins within cells was assessed. Hotspot regions within cells that are prone to carbonylation. Confocal microscopy imaging of 2b-modified fixed T-47D cells was conducted at concentrations of 250 μM and 1 mM. No fluorescence signal was observed in the HA-647 channel for unmodified cells. However, a dose-dependent fluorescent intensity (250 μM and 1 mM) was observed for HA-647 cells treated with carbonylation reaction conditions. This result also identified the localization of chemically carbonylated proteins within the cell membrane, cytoplasmic, and nuclear regions, indicating the potential of this method for carbonylation of proteins within diverse spatiotemporal localizations within a cell.

### Carbonylation Mediated Installation of Post-Translational Modifications

It is contemplated that a broader applicability of the carbonylation platform can be implemented utilizing the aldehyde moiety generated through carbonylation which can be used as a reactive handle for incorporating neo-post-translational modifications (PTMs) onto proteins. Post-translational modifications (PTMs) of proteins enhance their structural and functional diversity. A broad range of chemically plausible side chains, both natural and unnatural, is difficult to access through conventional methods. A carbonylation strategy was utilized that converts arginine residues in proteins into aldehydes and can serve as a flexible platform for introducing a wide variety of PTMs, both natural and synthetic. For example, myoglobin was modified using optimized reaction conditions with probe 2b to introduce a single aldehyde handle on the protein. Homogeneously modified myoglobin was then subjected to reductive amination conditions. By employing ammonia, N-methylamine, N,N-dimethyl amine, N-methyl propargylamine, and N,N-diethyl amine, on can generate ornithine and incorporated other diverse PTMs on the side chain of ornithine, such as monomethylation, dimethylation, alkynylation, and diethylation. Products were derived from a single, readily accessible aldehyde precursor on the protein through reductive amination. These modifications promote unique bonding patterns that can expand biological functions beyond those typical in natural proteins. This approach to chemically editing arginine residues bypasses the rigid constraints of ribosomal and enzymatic processes. As there are limited chemical methods available to selectively introduce these natural and unnatural PTMs onto specific amino acids on proteins in a targeted manner these compositions and methods provide a significant advancement in the ability to expand the functional repertoire of proteins.

1. A method of oxidizing a guanidine group comprising contacting a compound having a guanidine group with a 9,10-phenanthrenequinone or derivative and a base providing compound with an aldehyde group in place of the guanidine group.

2. The method of claim 1, wherein the guanidine group is an arginine amino acid within a protein.

3. The method of claim 1 wherein the base is 1,8-diazabicycloundec-7-ene (DBU) or potassium phosphate base.

4. The method of claim 3, wherein the compound with an aldehyde group is contacted with a labeling agent comprising an amine group.

5. The method of claim 4, wherein the amine group is within a primary amine group, secondary amine group, hydroxy amine group, hydrazine group, aniline amine group.

6. The method of claim 4, wherein labeling agent comprises an aromatic molecule, a fluorescent agent, a ligand, biotin, a receptor, enzyme, an antigen, radioisotope, or an antibody.

7. The method of claim 1, wherein the 9,10-phenanthrenequinone or derivative has the following formula I, 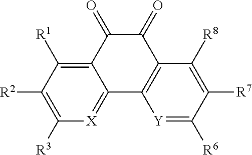
or salt thereof, wherein
X is N or CR⁴;
Y is N or CR⁵;
R¹, R², R³, R⁴, R⁵, R⁶, R⁷ and R⁸ are individually selected from hydrogen, C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein each R¹, R², R³, R⁴, R⁵, R⁶, R⁷ and R⁸ are optionally substituted with one or more, the same or different, R⁹; and
R⁹ is C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein Rois optionally substituted with one or more, the same or different, R¹⁰; or
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, isopropoxycarbonyl, tert-butoxycarbonyl, benzoyl, benzyl, carbocyclyl, aryl, or heterocyclyl.

8. The method of claim 7, wherein R³ is carboxy.

9. The method of claim 7, wherein R⁴ is nitro and R⁷ is nitro.

10. The method of claim 7, wherein X is N and Y is N.

11. The method of claim 7, wherein the 9,10-phenanthrenequinone is 9,10-phenanthrenequinone-3-carboxylic acid or salt thereof.

12. The method of claim 7, wherein the 9,10-phenanthrenequinone is 1,10-phenanthroline-5,6-dione or salt thereof.

13. The method of claim 7, wherein the 9,10-phenanthrenequinone is 2,5-dinitrophenanthrene-9,10-dione.

14. A composition comprising a 9,10-phenanthrenequinone derivative having the following formula I, 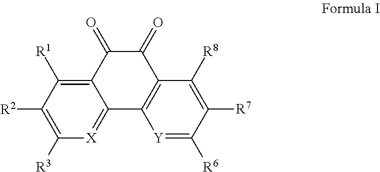
or salt thereof, wherein
X is N or CR⁴;
Y is N or CR⁵;
R¹, R², R³, R⁴, R⁵, R⁶, R⁷ and R⁸ are individually selected from hydrogen, C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein each R¹, R², R³, R⁴, R⁵, R⁶, R⁷ and R⁸ are optionally substituted with one or more, the same or different, R⁹; and
R⁹ is C₁₋₁₀ alkyl, C₂₋₁₀ alkenyl or C₂₋₁₀ alkynyl, halogen, nitro, cyano, hydroxy, amino, mercapto, formyl, carboxy, carbamoyl, alkoxy, hydroxyalkyl, glycol, alkylthio, thioalkyl, alkylamino, aminoalkyl, (alkyl)₂amino, alkanoyl, alkoxycarbonyl, carbocyclyl, benzoyl, benzyl, aryl, or heterocyclyl, wherein R⁹ is optionally substituted with one or more, the same or different, R¹⁰; or
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, isopropoxycarbonyl, tert-butoxycarbonyl, benzoyl, benzyl, carbocyclyl, aryl, or heterocyclyl.

15. The composition of claim 14, wherein R⁶ is carboxy.

16. The composition of claim 15, wherein the compound is 9,10-phenanthrenequinone-3-carboxylic acid or salt thereof.

17. The composition of claim 14, wherein the compound is in cell culture medium.

18. The composition of claim 14, wherein the compound is in a cell or cell lysate.

19. The composition of claim 14, wherein further comprising a label substituted with primary amine or hydroxy amine group.

20. The composition of claim 19, wherein the label is a fluorescent dye.
